(19) United States
(12) Patent Application Publication
(10) Pub. No.: US 2007/0276168 A1
(43) Pub. Date: Nov. 29, 2007

(54) Method For Preparation Of A Fluoroaromatic Compound From An Aminoaromatic Compound

(76) Inventors: Laurent Garel, Lyons (FR); Laurent Saint-Jalmes, Vourles (FR)

Correspondence Address: Jean-Louis Seugnet;RHODIA, 8 Cedar Brook Drive, CN 7500, Cranbury, NJ, 08512-7500, US

(21) Appl. No.: 10/588,471

(22) PCT Filed: Feb. 3, 2005
(86) PCT No.: PCT/FR05/00238

§ 371 Date: Jun. 20, 2007

(30) Foreign Application Priority Data
Feb. 5, 2004 (FR) 0401105

(51) Int. Cl. C07C 25/13

(52) U.S. Cl. 570/141

(57) ABSTRACT

The invention relates to a method for preparation of fluorinated aromatic compounds from the corresponding amines by replacement of the amino group with a fluorine atom. The method of preparation of a fluoroaromatic compound from an aromatic compound with at least one amino group on the aromatic ring is characterized by the reaction of said aromatic amine compound with a nitrosating agent in the presence of a source of boron trifluoride in an organic medium, and carrying out a thermal treatment of the reaction medium comprising the diazonium salt thus obtained to give the fluoroaromatic compound directly by decomposition of the diazonium salt without intermediate isolation thereof.

A subject matter of the present invention is a process for the preparation of fluorinated aromatic compounds from corresponding amines by replacement of the amino group by a fluorine atom.

Brominated or chlorinated aromatic compounds are easily obtained by electrophilic halogenation with molecular bromine or chlorine but fluoroaromatic compounds are, for their part, much more rarely synthesized directly by fluorination with fluorine; this reaction being difficult to control.

Two methods for the introduction of fluorine have been developed.

The first consists in substituting a halogen atom by a fluoride by means of the chlorine-by-fluorine halogen-exchange process [B. Langlois, L. Gilbert and G. Forat, Ind. Chem. Libr., 1996, 8, 244]. This exchange is highly suitable for chlorinated (or brominated) substrates for which electron-withdrawing groups are situated in the ortho and/or in the para position with respect to the halogen. Furthermore, groups such as NO₂ can be displaced (fluorodenitration).

The second method consists in replacing a diazonium group N₂⁺ by a fluorine. It is generally carried out in two stages: a diazotization reaction followed by a fluoro-dediazotization.

Thus, an aniline can be diazotized with sodium nitrite in anhydrous hydrofluoric acid and the arenediazonium fluoride thus obtained is subjected to thermal decomposition to give a fluoroaromatic compound. This reaction is used for simple fluoroaromatics (fluorobenzene, 3-fluorotoluene), [N. Ishikawa, Petrotech, 1987, 10, 543]. This reaction sequence exhibits the disadvantage of not being suitable for aminoarenes possessing chemically unstable groups (nitrile, ketone, and the like) and requires specific equipment. In order to minimize the formation of tar, it has been recommended, by N. Yoneda [Tetrahedron, 1991, 47, 5329], to add bases to the hydrofluoric acid but, generally, the productivity output is lower than in hydrofluoric acid alone.

This route employing hydrofluoric acid requires special equipment and is of restricted application as it is suitable only for substrates not possessing groups unstable chemically towards acidic conditions.

Another, older, diazotization method consists in carrying out the diazotization of the aminoarene in an aqueous medium with hydrochloric acid and sodium nitrite. The arenediazonium chloride formed is soluble in the medium but the addition of an aqueous solution of sodium tetrafluoroborate or of fluoroboric acid results in the precipitation of a diazonium tetrafluoroborate formed.

Aromatic amines can also be diazotized directly in aqueous tetrafluoroboric acid or in aqueous hydrofluoric acid into which boron trifluoride is introduced.

The arenediazonium tetrafluoroborate obtained is subjected to a fluoro-dediazotization operation by heating until it decomposes to give a fluoroaromatic compound, nitrogen and boron trifluoride. However, this “Balz-Schiemann” reaction is highly exothermic.

The synthesis of fluoroaromatic compounds in aqueous medium and from anilines exhibits numerous disadvantages.

The productive output by volume is sometimes not very high due to the low solubility of some amines in an aqueous medium.

When the diazotization is carried out in aqueous medium with hydrochloric acid and sodium nitrite, chlorinated impurities may be formed during the fluoro-dediazotization reaction. Furthermore, this process generates large amounts of saline aqueous effluents which have to be treated.

It can be dangerous to dry and isolate the arenediazonium tetrafluoroborate (thermal runaway, explosions, toxicity, and the like). In point of fact, however, the drying is necessary insofar as arenediazonium tetrafluoroborates are less stable, decompose at a lower temperature and decompose more violently when they are wet. Furthermore, the presence of water can result in phenols.

The object of the present invention is to provide a process which makes it possible to overcome the abovementioned disadvantages.

There has now been found, and it is this which constitutes the subject matter of the present invention, a process for the preparation of a fluoroaromatic compound from an aromatic compound carrying at least one amino group on the aromatic ring, characterized in that said aminoaromatic compound is reacted with a nitrosating agent, in the presence of a source of boron trifluoride, in an organic medium, and in that a heat treatment is carried out on the reaction medium comprising the diazonium salt obtained, thus making it possible to directly access the fluoroaromatic compound, by decomposition of the diazonium salt, without intermediate separation of the latter.

In the present text, the term “decomposition temperature of the diazonium salt” is understood to mean the temperature of conversion of the diazonium salt to give a fluoroaromatic compound, determined by differential thermal analysis on a preprepared sample.

Thus, the process of the invention makes it possible to directly access the fluoroaromatic compound, by decomposition of the diazonium salt, without intermediate separation of the latter.

According to a preferred embodiment of the process of the invention, the process consists in carrying out the decomposition of the diazonium salt formed in the reaction medium as it is formed.

The process of the invention thus makes it possible to avoid the safety problems related to the handling of a diazonium salt.

Thus, according to this preferred embodiment, the process for the preparation of a fluoroaromatic compound according to the invention comprises the following sequences:
the source of boron trifluoride, the aromatic compound carrying at least one amino group on the aromatic ring and the organic solvent are mixed by introducing in any order,
the reaction medium is brought to the decomposition temperature of the diazonium salt,
the nitrosating agent is gradually added,
the fluoroaromatic compound formed is recovered.

Another embodiment consists in introducing the reactants in a different order.

Thus, the source of boron trifluoride, the nitrosating agent and the organic solvent are mixed by introducing in any order; the reaction medium is brought to the decomposition temperature of the diazonium salt; the aromatic compound carrying at least one amino group on the aromatic ring is gradually added and then the fluoroaromatic compound formed is recovered.

In these preferred embodiments, the process of the invention makes it possible to avoid the accumulation of the diazonium salt in the reaction medium.

In accordance with the process of the invention, a diazonium salt is prepared as an intermediate by reaction of an aromatic compound carrying at least one amino group on the aromatic ring and a nitrosating agent, in the presence of a source of boron trifluoride, in an organic medium, and said diazonium salt is decomposed without intermediate separation.

In the account which follows of the present invention, the term “aminoaromatic compound” is understood to mean an aromatic compound in which a hydrogen atom bonded directly to the aromatic nucleus is replaced respectively by an amino group and the term “aromatic compound” is understood to mean the conventional notion of aromaticity as defined in the literature, in particular by Jerry March, Advanced Organic Chemistry, 4th edition, John Wiley and Sons, 1992, pp. 40 et seq.

The invention relates more particularly to the aminoaromatic compounds corresponding to the general formula: 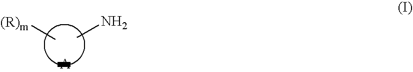
in said formula:
A symbolizes the residue of a ring forming all or part of a monocyclic or polycyclic, aromatic, carbocylic or heterocyclic system,
R, which are identical or different, represent substituents on the ring,
m represents the number of substituents on the ring.

The invention applies in particular to the aminoaromatic compounds corresponding to the formula (I) in which A is the optionally substituted residue of a cyclic compound preferably having at least 4 atoms in the ring, preferably 5 or 6, and representing at least one of the following rings:
a monocyclic or polycyclic, aromatic, carbocycle,
a monocyclic or polycyclic, aromatic, heterocycle comprising at least one of the heteroatoms O, N and S.

It will be specified, without, however, limiting the scope of the invention, that the optionally substituted residue A represents the residue:

1°—of a monocyclic or polycyclic, aromatic, carbocyclic compound.
The term “polycyclic carbocyclic compound” is understood to mean:
   a compound composed of at least 2 aromatic carbocycles forming, between them, ortho- or ortho- and peri-fused systems,
   a compound composed of at least 2 carbocycles, one alone of which is aromatic, forming, between them, ortho- or ortho- and peri-fused systems.

2°—of a monocyclic or polycyclic, aromatic, heterocyclic compound.
The term “polycyclic heterocyclic compound” defines:
   a compound composed of at least 2 heterocycles comprising at least one heteroatom in each ring, at least one of the two rings of which is aromatic, forming, between them, ortho- or ortho- and peri-fused systems,
   a compound composed of at least one carbocycle and at least one heterocycle, at least one of the rings of which is aromatic, forming, between them, ortho- or ortho- and peri-fused systems.

3°—of a compound composed of a series of rings, as defined in paragraphs 1 and/or 2, bonded to one another:
via a valency bond,
via an alkylene or alkylidene group having from 1 to 4 carbon atoms, preferably a methylene or isopropylidene group,
via one of the following groups: 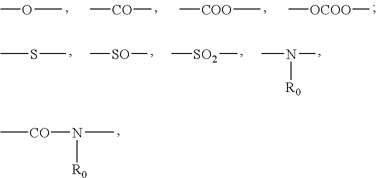
in these formulae, R₀ representing a hydrogen atom or an alkyl group having from 1 to 4 carbon atoms, a cyclohexyl group or a phenyl group.

By way of examples, the optionally substituted residue A represents one of the following rings:
an aromatic carbocycle: 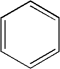
an aromatic bicycle comprising two aromatic carbocycles: 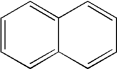
a partially aromatic bicycle comprising two carbocycles, one of the two of which is aromatic: 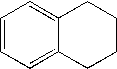
an aromatic heterocycle: 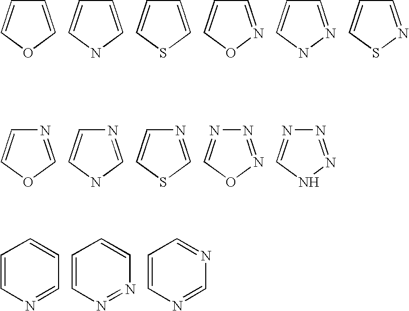
an aromatic bicycle comprising an aromatic carbocycle and an aromatic heterocycle: 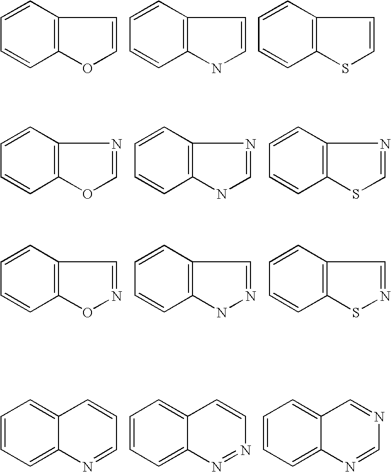
a partially aromatic bicycle comprising an aromatic carbocycle and a heterocycle: 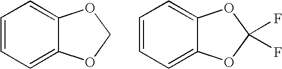
an aromatic bicycle comprising two aromatic heterocycles:

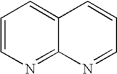

a partially aromatic bicycle comprising a carbocycle and an aromatic heterocycle: 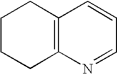
a tricycle comprising at least one aromatic carbocycle or heterocycle: 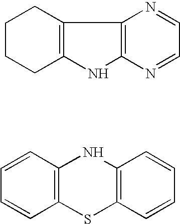
a series of aromatic carbocycles: 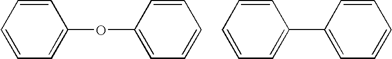
a partially aromatic series of carbocycles: 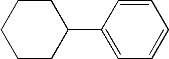
a series of an aromatic carbocycle and of an aromatic heterocycle: 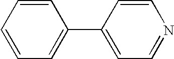
a partially aromatic series of a carbocycle and of a heterocycle:

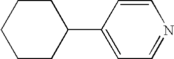

In the process of the invention, use is preferably made of an aminoaromatic compound of formula (I) in which A represents an aromatic nucleus, preferably a benzene, naphthalene, pyridine or quinoline nucleus.

The aromatic compound of formula (I) can carry one or more substituents.

The number of substituents present on the ring depends on the carbon fusion of the ring and on the presence or absence of unsaturations in the ring.

The maximum number of substituents capable of being carried by a ring is easily determined by a person skilled in the art.

In the present text, the term “several” is understood to mean, generally, less than 4 substituents on an aromatic nucleus.

Examples of substituents are given below but this list does not exhibit a limiting nature.

The group or groups R, which are identical or different, preferably represent one of the following groups:
a linear or branched alkyl group having from 1 to 6 carbon atoms, preferably from 1 to 4 carbon atoms, such as methyl, ethyl, propyl, isopropyl, butyl, isobutyl, sec-butyl or tert-butyl,
a linear or branched alkenyl or alkynyl group having from 2 to 6 carbon atoms, preferably from 2 to 4 carbon atoms, such as vinyl or allyl,
a linear or branched alkoxy group having from 1 to 6 carbon atoms, preferably from 1 to 4 carbon atoms, such as the methoxy, ethoxy, propoxy, isopropoxy or butoxy groups, an alkenyloxy group, preferably an allyloxy group, or a phenoxy group,
a cyclohexyl, phenyl or benzyl group,
an acyl group having from 2 to 6 carbon atoms,
a group of formula:
   —R₁—OH
   —R₁—SH
   —R₁—COOM
   —R₁—COOR₂
   —R₁—CO—R₂
   —R₁—CHO
   —R₁—N═C═O
   —R₁—N═C═S
   —R₁—NO₂
   —R₁—CN
   —R₁—N(R₂)₂
   —R₁—CO—N(R₂)₂
   —R₁—SO₃M
   —R₁—SO₂M
   —R₁—X
   —R₁—CF₃
   —R₁—CₚF₂ₚ₊₁
in said formulae, R₁ represents a valency bond or a saturated or unsaturated and linear or branched divalent hydrocarbon group, having from 1 to 6 carbon atoms, such as, for example, methylene, ethylene, propylene, isopropylene or isopropylidene; the groups R₂, which are identical or different, represent a hydrogen atom or a linear or branched alkyl group having from 1 to 6 carbon atoms or a phenyl group; M represents a hydrogen atom, an alkali metal, preferably sodium, or a group R₂; X symbolizes a halogen atom, preferably a chlorine, bromine, fluorine or iodine atom; p represents a number ranging from 1 to 10.

The present invention applies very particularly to the compounds corresponding to the formula (I) in which the group or groups R represent:
a linear or branched alkyl group having from 1 to 6 carbon atoms, preferably from 1 to 4 carbon atoms, such as methyl, ethyl, propyl, isopropyl, butyl, isobutyl, sec-butyl or tert-butyl,
a linear or branched alkenyl group having from 2 to 6 carbon atoms, preferably from 2 to 4 carbon atoms, such as vinyl or allyl,
a linear or branched alkoxy group having from 1 to 6 carbon atoms, preferably from 1 to 4 carbon atoms, such as the methoxy, ethoxy, propoxy, isopropoxy or butoxy groups, an alkenyloxy group, preferably an allyloxy group, or a phenoxy group,
a group of formula:
   —R₁—OH
   —R₁—N(R₂)₂
   —R₁—SO₃M
in said formulae, R₁ represents a valency bond or a saturated or unsaturated and linear or branched divalent hydrocarbon group having from 1 to 6 carbon atoms, such as, for example, methylene, ethylene, propylene, isopropylene or isopropylidene; the groups R₂, which are identical or different, represent a hydrogen atom or a linear or branched alkyl group having from 1 to 6 carbon atoms or a phenyl group; M represents a hydrogen atom or a sodium atom.

In the formula (I), m is a number less than or equal to 4, preferably equal to 1 or 2. As regards p, it is preferably equal to 1 or 2.

The invention relates more particularly to the aromatic carbocyclic compounds of following formula (Ia): 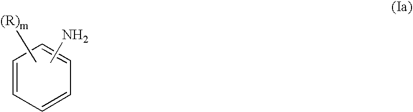
in said formula:
R and m have the meaning given above.

The invention is preferably targeted at the nitrogenous heterocyclic compounds corresponding to the following formula (Ib): 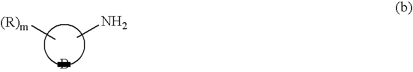
in said formula:
R and m have the meaning given above,
B represents the residue of a monocyclic aromatic heterocycle comprising 5 or 6 atoms, one or two of which among them are nitrogen atoms, or the residue of a polycyclic heterocycle comprising, on the one hand, an aromatic heterocycle comprising 5 or 6 atoms, one or two of which among them are nitrogen atoms, and, on the other hand, a carbocycle or a nitrogenous heterocycle which is saturated, unsaturated or aromatic and which comprises 5 or 6 atoms.

Mention may in particular be made, as examples of compounds corresponding to the formula (I), of the following aminoaromatic compounds: 4-bromoaniline, 4-bromo-3-methylaniline, 1-aminonaphthalene, 2-chloro-3-aminopyridine, 3-aminoquinoline or 3-amino-6-methoxy-quinoline.

Recourse is had, as diazotizing reagent, to any proton-free NO⁺-generating nitrosating agent.

Thus, it is possible to start from nitrogen dioxide NO₂, nitrogen trioxide N₂O₃ or nitrogen tetroxide N₂O₄. Should the reagent be a gas under the reaction conditions, it is bubbled into the medium.

It is also possible to employ alkyl nitrites and more particularly those corresponding to the formula (II):
Rₐ—ONO (II)
in said formula (II), Rₐ represents a linear or branched alkyl group having from 1 to 12 carbon atoms, preferably from 1 to 6 carbon atoms, or a cycloalkyl group having 5 or 6 carbon atoms.

The choice is advantageously made of n-butyl, tert-butyl or isoamyl nitrite.

As regards the source of boron trifluoride, recourse may be had to BF₃ in the gaseous form.

However, it is preferable to resort to boron trifluoride complexes comprising approximately between 20 and 70% by weight of boron trifluoride.

Mention may in particular be made, as examples of complexes, of the complexes comprising boron trifluoride in combination with an organic compound of Lewis base type chosen from water, ethers, alcohols and phenols, acetic acid or acetonitrile.

Mention may in particular be made, as examples of ethers, of dimethyl ether, diethyl ether, dibutyl ether or methyl tert-butyl ether.

Mention may be made, as other solvents, inter alia, of alcohols, such as methanol, propanol or phenol.

Recourse is preferably had to commercially available sources of boron trifluoride.

Mention may in particular be made of the BF₃.2H₂O complex or the complexes of BF₃ and of acetic acid, of diethyl ether, of dibutyl ether or of methyl tert-butyl ether.

The choice is preferably made, as preferred reagents, of boron trifluoride in combination with water, acetic acid or diethyl ether.

In accordance with the process of the invention, the reaction is carried out in an organic medium, which means that there is present an organic solvent or optionally a mixture of organic solvents.

The choice of the solvent is such that it must not exhibit a reducing nature with respect to the diazonium salt.

Recourse is had to a polar or nonpolar aprotic solvent.

Mention may be made, as nonlimiting examples of solvents suitable in the process of the invention, of:
aliphatic hydrocarbons and more particularly paraffins, such as, in particular, pentane, hexane, heptane, octane, isooctane, nonane, decane, undecane, tetradecane, petroleum ether and cyclohexane; aromatic hydrocarbons, such as, in particular, benzene, toluene, xylenes, ethylbenzene, diethylbenzenes, trimethylbenzenes, cumene, pseudocumene, petroleum fractions composed of mixtures of alkylbenzenes, in particular fractions of Solvesso® type,
halogenated aliphatic or aromatic hydrocarbons, and mention may be made of: perchlorinated hydrocarbons, such as, in particular, trichloromethane or tetrachloroethylene; partially chlorinated hydrocarbons, such as dichloromethane, dichloroethane, tetrachloroethane, trichloroethylene, 1-chlorobutane, 1,2-dichlorobutane; monochlorobenzene, 1,2-dichlorobenzene, 1,3-dichlorobenzene, 1,4-dichlorobenzene or mixtures of different chlorobenzenes; perfluorodecalin, trifluoromethylbenzene,
aliphatic, cycloaliphatic or aromatic ethers and more particularly methyl tert-butyl ether, dipentyl ether, diisopentyl ether, ethylene glycol dimethyl ether (or 1,2-dimethoxyethane), diethylene glycol dimethyl ether (or 1,5-dimethoxy-3-oxapentane) or cyclic ethers, for example dioxane or tetrahydrofuran,
aliphatic or aromatic nitrites, such as acetonitrile, propionitrile, butanenitrile, isobutanenitrile, benzonitrile or benzyl cyamide,
linear or cyclic carboxamides, such as N,N-dimethylacetamide (DMAC), N,N-diethylacetamide, dimethylformamide (DMF) or diethylformamide,
N-methylpyrrolidone.

Among all these solvents, chlorobenzene, 1,2-dichlorobenzene, toluene and benzonitrile are preferred.

As regards the amounts of reagents and the conditions for carrying out the process of the invention, those which are preferred are specified below.

The amount of diazotizing reagent employed can vary widely. When it is expressed by the aminoaromatic compound/nitrosating agent defined as NO⁺ molar ratio, it is at least equal to the stoichiometric amount but it is preferable for it to be employed in an excess which can reach 120% of the stoichiometric amount and preferably of between 100% and 120%.

The amount of the fluoride source employed is such that the F⁻/aminoaromatic compound molar ratio varies between 1 and 2, preferably between 1.2 and 1.5.

As regards the amount of organic solvent employed, it is such that the concentration of the aminoaromatic substrate in the reaction medium is preferably between 0.5 and 2.5 mol/l and is preferably in the vicinity of 1 mol/l.

It should be noted that the process of the invention resorts to a nonionic source of boron trifluoride.

As regards the temperature and pressure conditions, they are advantageously as described below.

The diazotization reaction of the first stage is generally carried out at a low temperature advantageously lying between −10° C. and 20° C., preferably between 0 and 10° C.

As regards the decomposition temperature of the diazonium salt, it can vary between ambient temperature and 150° C., preferably between 40° C. and 130° C.

The term “ambient temperature” is understood to mean, generally, a temperature of between 15° C. and 25° C.

The duration of the heat treatment advantageously varies between 5 minutes and 4 hours, preferably between 15 minutes and 2 hours.

The process of the invention is carried out at atmospheric pressure but preferably under a controlled atmosphere of inert gases, such as nitrogen or rare gases, for example argon. A pressure slightly greater or less than atmospheric pressure may be suitable.

As regards the methods of implementing the invention in practice, one among them comprises the formation of a diazonium salt from the aminoaromatic compound and then the thermal decomposition of said salt in the reaction medium.

To this end, the source of boron trifluoride, the aminoaromatic compound and the organic solvent are introduced in any order; the nitrosating agent is introduced and the reaction medium is subjected to heat treatment in order to decompose the diazonium salt obtained without it being removed from the medium; the fluoroaromatic compound obtained is recovered.

More specifically, the source of boron trifluoride is charged, preferably in the form of a complex and at low temperature, for reasons of convenience of handling.

The temperature is advantageously chosen between −10° C. and 20° C., preferably between 0 and 10° C., with the exception of boron trifluoride in the dihydrate form, which is introduced at ambient temperature.

The aminoaromatic compound is subsequently added, all at once or gradually. Gradual addition is preferred.

There is no disadvantage to introducing first the aminoaromatic compound and then the source of fluoride.

The aminoaromatic compound can be introduced alone or in solution in all or part of the organic solvent employed in an amount representing, for example, from 50 to 100% by weight of the total amount of solvent involved.

The nitrosating agent is subsequently added, all at once or gradually. Gradual addition is preferred.

The nitrosating agent can be introduced alone or in solution in the organic solvent involved, for example between 0 and 50% by weight.

An arenediazonium or heteroarenediazonium salt is obtained and precipitates.

It preferably corresponds to the following formula (III): 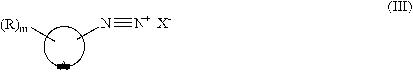
in which:
A, R and m have the meanings given above,
it being possible for X to be a salt derived from BF₃ or an alkoxide part (Rₐ—O⁻) originating from the alkyl nitrite.

The reaction medium is subjected to heat treatment in order to decompose the diazonium salt obtained without it being removed from the medium.

Heating is carried out in the temperature region defined above, namely between ambient temperature and 150° C., preferably between 40° C. and 130° C.

The fluoroaromatic compound (IV), which corresponds to the formula: 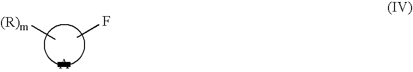
in which A, R and m have the meanings given above, is obtained.

It should be noted that the formula (IV) includes the compounds obtained from the aminoaromatic compounds corresponding to the formulae (Ia) and (Ib).

The fluoroaromatic compound is obtained in organic solution.

It is recovered according to conventional separating techniques, preferably by distillation.

According to a preferred embodiment, the process of the invention is carried out by decomposing the diazonium salt as it is formed in the reaction medium.

The use of the substrate, of the source of boron trifluoride and of the organic solvent is identical.

The source of boron trifluoride is charged, preferably at low temperature, and then the aminoaromatic compound, alone or in organic solution, is subsequently added, all at once or gradually. Gradual addition is preferred.

After addition of the aminoaromatic compound, heating is carried out in order to bring the reaction medium to the decomposition temperature of the diazonium salt, the temperature being chosen between ambient temperature and 150° C., preferably between 40° C. and 130° C.

In a following sequence, the nitrosating agent, preferably an alkyl nitrite, is added to the reaction medium maintained at the decomposition temperature of the diazonium salt.

The duration of the heat treatment, which comprises the rise in temperature and the addition of the third reagent, advantageously varies between 5 minutes and 4 hours, preferably between 15 minutes and 2 hours.

The reaction is continued until evolution of gas has completely ceased (nitrogen, possibly boron trifluoride). The heating can be continued, to this end.

An arenediazonium or heteroarenediazonium salt which preferably corresponds to the formula (III) is obtained as an intermediate, this salt being present in the reaction medium only in a very small amount since it is rapidly decomposed. The order of magnitude of the concentration of diazonium salt of approximately ten times lower than the concentration of starting aminoaromatic compound will be specified, by way of indication.

At the end of the reaction, the fluoroaromatic compound preferably corresponding to the formula (IV) is obtained in organic solution.

It is recovered conventionally, as described above.

According to another embodiment of the process of the invention, the source of boron trifluoride, the nitrosating agent and the organic solvent are introduced in any order, then the mixture is heated to the decomposition temperature of the diazonium salt and, subsequently, the aminoaromatic compound is introduced.

The latter is added gradually. The addition can be carried out portionwise or continuously.

A compound of formula (IV) is obtained and is recovered as described above.

The process of the invention is easily carried out according to a continuous process.

The process of the invention is particularly advantageous because it makes it possible to obtain fluoroaromatic compounds which are difficult to access, in particular due to the presence of unstable groups (for example, CO), or fluorinated nitrogenous heterocyclic compounds.

The process of the invention exhibits numerous advantages.

It makes it possible to save on a stage of separating the diazonium salt.

It corresponds better to safety requirements because the diazonium salt is not isolated and preferably decomposed as it is formed, which reduces the risks of explosion or of thermal runaway.

It is less polluting than the prior processes because it does not use any source of acid other than BF₃, which, by neutralization, results in the formation of inorganic salts which can be easily isolated.

Implementational examples of the invention are given below by way of illustration and without a limiting nature.

In the examples, the abbreviations used are defined thus:

The degree of conversion (DC) corresponds to the ratio of the number of moles of substrate converted to the number of moles of substrate involved.

The yield (RY) corresponds to the ratio of the number of moles of product formed to the number of moles of substrate involved.

### EXAMPLES

### Example 1

### Preparation of m-fluorotoluene

534 mg (4.98 mmol) of m-toluidine in 2.96 g of o-dichlorobenzene (o-DCB) are slowly introduced over 5 min onto a heel of 1.05 g (7.42 mmol, 1.5 mol. eq.) of BF₃.Et₂O at a temperature of −15° C. in a three-necked round-bottomed flask equipped with a reflux condenser, a thermocouple and a stirring system.

745 mg (6.49 mmol) of t-butyl nitrite in 1.96 g of o-DCB are then added dropwise at this temperature (or at ambient temperature) and then the reaction medium is heated at 100° C. for 17 min.

The reaction yield, determined by gas chromatography (GC) and ¹⁹F NMR, is 60%, the degree of conversion being 100%.

### Examples 2 to 6

Before describing the examples in detail, the procedure which is used in all the examples is specified.

An aminoaromatic compound in a solvent (o-dichlorobenzene or benzonitrile) is slowly introduced onto a BF₃.Et₂O heel (1.4-1.5 molar equivalents) at a temperature of less than 0° C. or onto a BF₃.2H₂O heel (1.4-1.5 molar equivalents) at ambient temperature in a three-necked round-bottomed flask equipped with a reflux condenser, a thermocouple and a stirring system.

t-Butyl nitrite in the same solvent is then added at ambient temperature and then the reaction medium is heated at the temperature specified in the table below and according to the time shown.

The results are recorded in table (I). Table I

TABLE I

| | Amino- | | | | | T (° C.) | |
|---|---|---|---|---|---|---|---|
| | aromatic | [C] | | | | Balz- | |
| Ex. | compound | A.C.* | ^{t}BuONO | BF₃.Et₂O | | Schiemann | Results |
| ref. | (1.0 mol. eq.) | (mol/l) | (mol. eq.) | (mol. eq.) | Solvent | reaction | (%) |
| 2 | 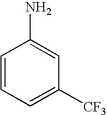 | 0.6 | 1.3 | 1.4 | o-DCB | 90° C./ 40 min | DC_{m-TFMA} = 100 RY = 44 |
| 3 | 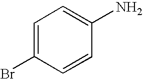 | 0.75 | 1.3 | 1.5 | o-DCB | 100° C./ 35 min 115° C./ 1.5 h | DC_{4-BA} = 100 RY = 54 |
| 4 | 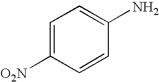 | 0.51 | 1.2 | 1.5 | Ph—CN | 110° C./ 20 min 120° C./ 45 min | DC_{4-NA} = 100 RY = 25 |
| 5 | 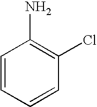 | 0.91 | 1.3 | 1.5 | o-DCB | 110° C./ 20 min 130° C./ 1 h | DC_{2-CIA} = 100 RY = 39 |
| 6 | 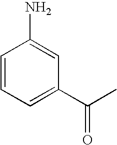 | 0.47 | 1.3 | 1.5 | Ph—CN | 90° C./ 45 min | DC_{3-AAcP} = 100 RY = 25 |

*A.C. = aminoaromatic compound

### Examples 7 and 8

The procedure of example 1 is reproduced according to the conditions defined in table (II).

The results are recorded in table (II).

TABLE II

| | Amino- | | | | | T (° C.) | |
|---|---|---|---|---|---|---|---|
| | aromatic | [C] | | | | Balz- | |
| Ex. | compound | A.C.* | ^{t}BuONO | BF₃.X | | Schiemann | Results |
| ref. | (1.0 mol. eq.) | (mol/l) | (mol. eq.) | (mol. eq.) | Solvent | reaction | (%) |
| 7 | 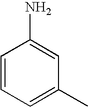 | 0.9 | 1.3 | 1.5 X = Et₂O | o-DCB | 100° C./ 20 min | DC_{m-Tol} = 100 RY_{3-FT} = 60 RY_{m-cresol} = 0 RYₜₒₗᵤₑₙₑ = 0 |
| 8 | | 0.94 | 1.4 | 1.5 X = 2H₂O | o-DCB | 100° C./ 20 min | DC_{m-Tol} = 100 RY_{3-FT} = 56 RY_{m-cresol} = 0 RYₜₒₗᵤₑₙₑ = 0 |

*A.C. = aminoaromatic compound

### Example 9

### Preparation of M-Fluorotoluene

284 mg (2.62 mmol) of m-toluidine in 3.03 g of o-dichlorobenzene are introduced dropwise over 6 minutes onto a heel of 400.2 mg (3.85 mmol, 1.47 mol. eq.) of BF₃.2H₂O at ambient temperature in a 25 ml three-necked flask equipped with a reflux condenser, a thermocouple and a stirring system.

At the end of the addition, the solution is pink with a precipitate.

The reaction medium is then heated to 100° C. and then, after 20 minutes, 388 mg (3.39 mmol, 1.29 mol. eq.) of t-butyl nitrite (purity 90%) in 2.02 g of o-dichlorobenzene are added at 100° C. with a flow rate of 5 ml/h.

When introduction of the t-butyl nitrite has begun, the reaction medium changes from pink to brown.

The temperature is maintained at 90° C. for 15 minutes and heating is halted.

The reaction yield of m-fluorotoluene, determined by GC and ¹⁹F NMR, is 39%.

### Example 10

### Preparation of 2-chloro-5-fluoropyridine

1 g (7.78 mmol) of 2-chloro-5-aminopyridine in 14.3 g of o-dichlorobenzene is introduced dropwise over 5 minutes onto a heel of 1.64 g (11.5 mmol, 1.47 mol. eq.) of BF₃.Et₂O at ambient temperature in a 25 ml three-necked flask equipped with a reflux condenser, a thermocouple and a stirring system.

At the end of the addition, the solution is beige with a precipitate.

The reaction medium is then heated to 105° C. and then, after 20 minutes, 1.05 g (9.2 mmol, 1.18 mol. eq.) of t-butyl nitrite (purity 90%) in 5.0 g of o-dichlorobenzene are added at this temperature with a flow rate of 25 ml/h.

The temperature is maintained at 105° C. for 25 minutes and heating is halted.

The reaction yield of 2-chloro-5-fluoropyridine, determined by ¹⁹F NMR, is 36%.

### Example 11

### Preparation of 3-fluoroquinoline

1 g (6.94 mmol) of 3-aminoquinoline in 10 ml of chlorobenzene is introduced dropwise over 10 minutes onto a heel of 0.66 ml (10.4 mmol, 1.5 mol. eq.) of BF₃.2H₂O at ambient temperature in a 50 ml three-necked flask equipped with a reflux condenser, a thermocouple and a stirring system.

The reaction medium is then heated to 50° C. and then 1.2 ml (9.01 mmol, 1.3 mol. eq.) of t-butyl nitrite (purity 90%) are added at this temperature over 30 minutes.

The reaction medium is brought to 100° C. and stirred for 1 hour.

The yield of isolated product is 40%.

### Example 12

### Preparation of 3-fluoro-6-methoxyquinoline

224 mg (1.29 mmol) of 3-amino-6-methoxyquinoline in 2.5 ml of o-dichlorobenzene are introduced dropwise over 5 minutes onto a heel of 125 μl (1.97 mmol, 1.5 mol. eq.) of BF₃.2H₂O at ambient temperature in a 25 ml three-necked flask equipped with a reflux condenser, a thermocouple and a stirring system.

The reaction medium is then heated to 40° C., then 0.22 ml (1.65 mmol, 1.3 mol. eq.) of t-butyl nitrite (purity 90%) in 0.3 ml of o-dichlorobenzene is added at this temperature over 7 min and then, after 40 min, the reaction medium is brought to 100° C. and stirred for 45 min.

The reaction yield, determined by ¹⁹F NMR, is 48% and the yield of isolated product is 34%.

1-35. (canceled)

36- A process for the preparation of a fluoroaromatic compound from an aromatic compound carrying at least one amino group on the aromatic ring, comprising the steps of:
a) reacting said aminoaromatic compound with a nitrosating agent, in the presence of a source of boron trifluoride, in an organic medium,
b) carrying out a heat treatment on the reaction medium comprising the diazonium salt obtained in step a), and thereby obtaining the fluoroaromatic compound, by decomposition of the diazonium salt, without intermediate separation of the latter, and
c) recovering the fluoroaromatic compound obtained in step b).

37- The process as claimed in claim 36, wherein in step a) the source of boron trifluoride, the aminoaromatic compound and the organic solvent are added in any order.

38- The process as claimed in claim 36, wherein the diazonium salt formed in the reaction medium is decomposed as it is formed.

39- The process as claimed in claim 38, wherein it comprises the following sequences:
the source of boron trifluoride, the aromatic compound carrying at least one amino group on the aromatic ring and the organic solvent are mixed by introducing in any order,
the reaction medium is brought to the decomposition temperature of the diazonium salt,
the nitrosating agent is gradually added, and
the fluoroaromatic compound formed is recovered.

40- The process as claimed in claim 38, wherein it comprises the following sequences:
the source of boron trifluoride, the nitrosating agent and the organic solvent are mixed by introducing in any order,
the reaction medium is brought to the decomposition temperature of the diazonium salt,
the aromatic compound carrying at least one amino group on the aromatic ring is gradually added, and
the fluoroaromatic compound formed is recovered.

41- The process as claimed in claim 36, wherein the source of boron trifluoride is charged at low temperature, the temperature being chosen between −10° C. and 20° C., optionally between 0 and 10° C., with the exception of boron trifluoride in the dihydrate form, which is added at ambient temperature.

42- The process as claimed in claim 36, wherein the aminoaromatic compound is added all at once or gradually.

43- The process as claimed in claim 36, wherein the aminoaromatic and/or the nitrosating agent compound is added alone or in solution in the organic solvent.

44- The process as claimed in claim 36, wherein the nitrosating agent is added all at once or gradually.

45- The process as claimed in claim 36, wherein the decomposition temperature of the diazonium salt varies between ambient temperature and 150° C., optionally between 40° C. and 130° C.

46- The process as claimed in claim 36, wherein the process is carried out at atmospheric pressure or under a controlled atmosphere of inert gases.

47- The process as claimed in claim 39, wherein the source of boron trifluoride is charged at low temperature; the aminoaromatic compound is gradually added; the reaction medium is heated to the decomposition temperature of the diazonium salt the nitrosating agent, optionally an alkyl nitrite, is gradually added.

48- The process as claimed in claim 47, wherein the reaction medium is heated to a temperature of between ambient temperature and 150° C., optionally between 40° C. and 130° C.

49- The process as claimed in claim 36, wherein the fluoroaromatic compound is recovered from the organic phase.

50- The process as claimed in claim 36, wherein the nitrosating agent is any proton-free NO⁺-generating source, a nitrogen dioxide NO₂, nitrogen trioxide N₂O₃, nitrogen tetroxide N₂O₄ or an alkyl nitrite.

51- The process as claimed in claim 16, wherein the alkyl nitrite corresponds to the formula (II):
Rₐ—ONO (II)
Wherein Rₐ represents a linear or branched alkyl group having from 1 to 12 carbon atoms, optionally from 1 to 6 carbon atoms, or a cycloalkyl group having 5 or 6 carbon atoms.

52- The process as claimed in claim 51, wherein the alkyl nitrite is n-butyl, tert-butyl or isoamyl nitrite.

53- The process as claimed in one of claims claim 36, wherein the source of boron trifluoride is boron trifluoride in the gaseous form or in the complex form.

54- The process as claimed in claim 53, wherein the source of fluoride is boron trifluoride in combination with a solvent chosen from water, ethers, alcohols and phenols, acetic acid or acetonitrile.

55- The process as claimed in claim 52, wherein the source of fluoride is boron trifluoride in combination with a solvent chosen from water, ethyl ether or acetic acid.

56- The process as claimed in claim 36, wherein the reaction is carried out in an organic solvent, optionally a polar or nonpolar aprotic solvent.

57- The process as claimed in claim 56, wherein the organic solvent is an halogenated or nonhalogenated aliphatic or an aromatic hydrocarbon; aliphatic, cycloaliphatic or aromatic ether, or a cyclic ethers; aliphatic or aromatic nitrite; linear or a cyclic carboxamide.

58- The process as claimed in claim 57, wherein the organic solvent is chlorobenzene, 1,2-dichlorobenzene, toluene, benzonitrile or N-methylpyrrolidone.

59- The process as claimed in claim 36, wherein the aminoaromatic compound corresponds to the general formula: 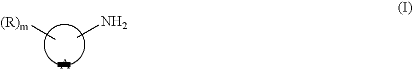
wherein:
A symbolizes the residue of a ring forming all or part of a monocyclic or polycyclic, aromatic, carbocylic or heterocyclic system,
R, which are identical or different, represent substituents on the ring,
m represents the number of substituents on the ring.

60- The process as claimed in claim 59, wherein the aminoaromatic compound corresponds to the formula (I) in which A is the optionally substituted residue of a cyclic compound optionally having at least 4 atoms in the ring, optionally 5 or 6, and representing at least one of the following rings:
a monocyclic or polycyclic, aromatic, carbocycle,
a monocyclic or polycyclic, aromatic, heterocycle comprising at least one of the heteroatoms O, N and S.

61- The process as claimed in claim 59, wherein the optionally substituted residue A represents the residue of:
a monocyclic or polycyclic, aromatic, carbocyclic compound,
a monocyclic or polycyclic, aromatic, heterocyclic compound,
in these formulae, R₀ representing a hydrogen atom or an alkyl group having from 1 to 4 carbon atoms, a cyclohexyl group or a phenyl group.

62- The process as claimed in claim 59, wherein the optionally substituted residue A represents:
an aromatic carbocycle,
an aromatic bicycle comprising two aromatic carbocycles,
a partially aromatic bicycle comprising two carbocycles, one of the two of which is aromatic,
an aromatic heterocycle,
an aromatic bicycle comprising an aromatic carbocycle and an aromatic heterocycle,
a partially aromatic bicycle comprising an aromatic carbocycle and a heterocycle,
an aromatic bicycle comprising two aromatic heterocycles,
a partially aromatic bicycle comprising a carbocycle and an aromatic heterocycle,
a tricycle comprising at least one aromatic carbocycle or heterocycle,
a series of aromatic carbocycles,
a partially aromatic series of carbocycles,
a series of an aromatic carbocycle and of an aromatic heterocycle, or
a partially aromatic series of a carbocycle and of a heterocycle.

63- The process as claimed in claim 59, wherein the optionally substituted residue A represents a benzene, naphthalene, pyridine or quinoline nucleus.

64- The process as claimed in claim 59, wherein the aminoaromatic compound corresponds to the formula (I) in which R, which are identical or different, represent:
a linear or branched alkyl group having from 1 to 6 carbon atoms, optionally from 1 to 4 carbon atoms, such as methyl, ethyl, propyl, isopropyl, butyl, isobutyl, sec-butyl or tert-butyl,
a linear or branched alkenyl or alkynyl group having from 2 to 6 carbon atoms, optionally from 2 to 4 carbon atoms, such as vinyl or allyl,
a linear or branched alkoxy group having from 1 to 6 carbon atoms, optionally from 1 to 4 carbon atoms, such as the methoxy, ethoxy, propoxy, isopropoxy or butoxy groups, an alkenyloxy group, optionally an allyloxy group, or a phenoxy group,
a cyclohexyl, phenyl or benzyl group,
an acyl group having from 2 to 6 carbon atoms,
a group of formula:
—R₁—OH
—R₁—SH
—R₁—COOM
—R₁—COOR₂
—R₁—CO—R₂
—R₁—CHO
—R₁—N═C═O
—R₁—N═C═S
—R₁—NO₂
—R₁—CN
—R₁—N(R₂)₂
—R₁—CO—N(R₂)₂
—R₁—SO₃M
—R₁—SO₂M
—R₁—X
—R₁—CF₃, or
—R₁—CₚF₂ₚ₊₁
in said formulae, R₁ represents a valency bond or a saturated or unsaturated and linear or branched divalent hydrocarbon group, having from 1 to 6 carbon atoms, such as, for example, methylene, ethylene, propylene, isopropylene or isopropylidene; the groups R₂, which are identical or different, represent a hydrogen atom or a linear or branched alkyl group having from 1 to 6 carbon atoms or a phenyl group; M represents a hydrogen atom, an alkali metal, optionally sodium, or a group R₂; X symbolizes a halogen atom, optionally a chlorine, bromine, fluorine or iodine atom; p represents a number ranging from 1 to 10.

65- The process as claimed in claim 59, wherein the aminoaromatic compound corresponds to the formula (I) in which m is a number less than or equal to 4, optionally equal to 1 or 2.

66- The process as claimed in claim 65, wherein the aminoaromatic compound corresponds to the formula (Ia): 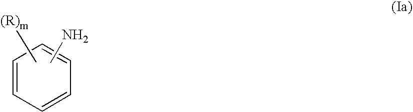
in said formula R and m have the meaning given above.

67- The process as claimed in claim 59, wherein the aminoaromatic compound corresponds to the formula (Ib): 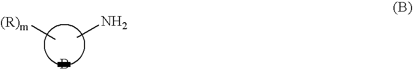
wherein:
R and m have the meaning given above,
B represents the residue of a monocyclic aromatic heterocycle comprising 5 or 6 atoms, one or two of which among them are nitrogen atoms, or the residue of a polycyclic heterocycle comprising, on the one hand, an aromatic heterocycle comprising 5 or 6 atoms, one or two of which among them are nitrogen atoms, and, on the other hand, a carbocycle or a nitrogenous heterocycle which is saturated, unsaturated or aromatic and which comprises 5 or 6 atoms.

68- The process as claimed in claim 59, wherein the aminoaromatic compound is chosen from: 4-bromoaniline, 4-bromo-3-methylaniline, 1-aminonaphthalene, 2-chloro-3-aminopyridine, 3-aminoquinoline or 3-amino-6-methoxyquinoline.
